(19) United States
(12) Patent Application Publication
(10) Pub. No.: US 2008/0069875 A1
(43) Pub. Date: Mar. 20, 2008

(54) Disintegrating Buccal Tablets

(75) Inventors: Yoshitomi Kakiguchi, Hyogo-ken (JP); Kunihiko Yokota, Osaka-fu (JP); Mitsunori Tanabe, Osaka-fu (JP); Tomoyoshi Kajiura, Mie-ken (JP); Tadaaki Tanaka, Chiba-ken (JP)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/662,483

(22) PCT Filed: Sep. 10, 2005
(86) PCT No.: PCT/EP05/09761

§ 371 Date: Mar. 9, 2007

(30) Foreign Application Priority Data
Sep. 13, 2004 (JP) 04/265216

(51) Int. Cl. A61K 9/20; A61K 47/30

(52) U.S. Cl. 424/464; 514/772.2

(57) ABSTRACT

Disintegrating buccal tablets comprising a physiologically active material and a polyvinyl alcohol/polyethylene glycol graft copolymer are disclosed along with methods of making the same.

The present inventions relate to disintegrating buccal tablets and to a method for the production thereof.

In order to facilitate penetration by saliva, etc, disintegrating buccal tablets need to be provided with porosity, and in order to accelerate their disintegration they need to be provided with swelling properties.

A major ingredient of disintegrating buccal tablets is a sugar and, in terms of taste and mouthfeel, etc, a sugar alcohol is often employed. This sugar generally lacks mouldability so there is incorporated a material such as crystalline cellulose having good mouldability and, furthermore, in order to achieve rapid disintegration there is employed carmellose sodium, croscarmellose sodium, carmellose calcium, hydroxypropyl cellulose with a low degree of substitution, crospovidone, starch or the like (see, for example, Patent Reference 1, JP-A-2003-81814 and Non-patent References 1 to 4: M. Sugihara et al: Research Report, Ministry of Health & Welfare Scientific Research GrantAided Silver Science Laboratory, Y. Honda et al: Byoin Yakugaku, 24(5), 533-540 (1998), Tsushima et al: Seizai Kikai Gijutsu Kenkyu Kaishi, 10(4), 305-317 (2001), K. Ogata et al: Iryo Yakugaku, 27(6), 553-558 (2001)).

Against this background, development is awaited of disintegrating buccal tablets in which novel types of additive are incorporated.

Now, polyvinyl alcohol/polyethylene glycol graft copolymer is used as a coating agent for tablets but it has not been used for disintegrating buccal tablets.

The present inventions have the objective of providing disintegrating buccal tablets in which a novel additive is incorporated.

As a result of painstaking research to meet this objective, it has been discovered that when disintegrating buccal tablets are produced using a polyvinyl alcohol/polyethylene glycol graft copolymer, which is a completely different type of additive from those conventionally used in disintegrating buccal tablets, quite unexpectedly the polyvinyl alcohol/polyethylene glycol graft copolymer functions as a disintegrating agent. The pre-sent inventions have been perfected based on this discovery.

The present inventions encompass the following.

(1) Disintegrating buccal tablets which contain a physiologically active material and a polyvinyl alcohol/polyethylene glycol graft copolymer.

(2) Disintegrating buccal tablets according to (1) above which contain a sugar.

(3) Disintegrating buccal tablets according to (2) above where the sugar is a sugar alcohol.

(4) A method for the production of disintegrating buccal tablets which is characterized in that after granulating a composition which contains a physiologically active material and a polyvinyl alcohol/polyethylene glycol graft copolymer, tableting is performed.

(5) A method according to (4) above where the aforesaid composition contains a sugar.

(6) A method according to (5) above where the sugar is a sugar alcohol.

(7) Disintegrating buccal tablets produced by a method as described in any of (4) to (6) above.

In accordance with the present inventions, it is possible to provide disintegrating buccal tablets in which a novel type of additive is incorporated.

The physiologically active material employed in the present inventions may have a solid form, a powder form, a crystalline form, an oily form, a solution form or the like, and there may be used one or more medicinal components selected from health supplements, antipyretic, analgesic and anti-inflammatory drugs, psychotropic drugs, antianxiety drugs, antidepressants, hypnotic sedatives, spasmolytic drugs, CNS-acting drugs, cerebral stimulants, cerebral circulation improvers, anticonvulsants, adrenergic agents, stomach medicines, antacids, anti-ulcer drugs, antitussive expectorants, antiemetics, respiratory stimulants, bronchodilators, antiallergics, dental and mouth drugs, antihistamines, cardiotonics, drugs for arrhythmia, diuretics, antihypertensives, vasoconstrictors, coronary (vaso) dilators, peripheral vasodilators, antihyperlipidemics, cholagogues, antibiotics, chemotherapeutic drugs, drugs for diabetes, drugs for osteoporosis, antirheumatics, skeletal muscle relaxants, hormone agents, alkaloid narcotics, sulphur drugs, gout remedies, anticoagulants, anticancer drugs and therapeutic agents for Alzheimer's disease. These physiologically active materials can be either in their free form or in the form of a salt, and they may be racemates or optically active isomers and, furthermore, they may also be prodrugs.

Examples of the health supplements are vitamins such as vitamin A, vitamin D, vitamin E (d-α-tocopherol acetate, etc), vitamin B₁ (dibenzoyl thiamine, fursultiamine hydrochloride, etc), vitamin B₂ (riboflavin butyrate, etc), vitamin B₆ (pyridoxine hydrochloride, etc), vitamin C (ascorbic acid, sodium L-ascorbate, etc), vitamin B₁₂ (hydroxocobalamin acetate, cyanocobalamin, etc), minerals such as calcium, magnesium and iron, proteins, amino acids, oligo sugars, herbal medicines and the like. Examples of the antipyretic, analgesic and anti-inflammatory drugs are aspirin, acetaminophen, ethenzamide, ibuprofen, diphenhydramine hydrochloride, di-chlorpheniramine maleate, dihydrocodeine phosphate, noscapine, methylephedrine hydrochloride, phenylpropanolamine hydrochloride, caffeine, anhydrous caffeine, serrapeptase, lysozyme hydrochloride, tolfenamic acid, mefenamic acid, diclofenac sodium, flufenamic acid, salicylamide, ketoprofen, indomethacin, bucolome, pentazocine and the like. Examples of the psychotropic drugs are chlorpromazine, reserpine and the like. Examples of the antianxiety drugs are alprazolam, chlordiazepoxide, diazepam and the like. Examples of the antidepressants are imipramine, maprotiline hydrochloride, amphetamine and the like. Examples of the hypnotic sedatives are estazolam, nitrazepam, diazepam, perlapine, sodium phenobarbital and the like. Examples of the spasmolytic drugs are scopolamine hydrobromide, diphenhydramine hydrochloride, papaverine hydrochloride and the like.

Examples of the CNS-acting drugs are citicoline and the like. Examples of the cerebral stimulants are meclofenoxate and the like. Examples of the cerebral circulation improvers are vinpocetine and the like. Examples of the anti-convulsants are phenyloin, carbamazepine and the like. Examples of the adrenergic drugs are isoproterenol and the like. Examples of the stomach medicines are diastase, sugar-containing pepsin, Scopolia extract, cellulase AP3, lipase AP, oil of cinnamon and other such stomachic digestive agents, berberine chloride, tolerant lactic acid bacteria, bifidus bacteria and other such agents for controlling intestinal function. Examples of the antacids are magnesium carbonate, sodium bicarbonate, magnesium metasilicate aluminate, synthetic hydrotalcite, precipitated calcium carbonate, magnesium oxide and the like. Examples of the anti-ulcer drugs are lansoprazole, omeprazole, rabeprazole, pantoprazole and other such benzimidazole type compounds or salts thereof (including optically active isomers) and other such PPIs, famotidine, cimetidine, ranitidine hydrochloride and other types of histamine H₂ receptor antagonist. Examples of the antitussive expectorants are cloperastine hydrochloride, dextromethorphan hydrobromide, theophylline, potassium guaiacol sulphonate, guaifenesin, codeine phosphate and the like. Examples of the antiemetics are difenidol hydrochloride, metoclopramide and the like. Examples of the respiratory stimulants are levallorphan tartrate and the like. Examples of the bronchodilators are theophylline, salbutamol sulphate and the like. Examples of the antiallergics are amlexanox, seratrodast and the like. Examples of the dental and mouth drugs are oxytetracycline, triamcinolone acetonide, chlorhexidine hydrochloride, lidocaine and the like. Examples of the antihistamines are diphenhydramine hydrochloride, promethazine, isothipendyl hydrochloride, dl-chlorpheniramine maleate and the like. Examples of the cardiotonics are caffeine, digoxin and the like. Examples of the drugs for arrhythmia are procainamide hydrochloride, propranolol hydrochloride, pindolol and the like. Examples of the diuretics are isosorbide, furosemide, HCTZ and other thiazide agents.

As examples of the antihypertensives, there are delapril hydrochloride, captopril, hexamethonium bromide, hydralazine hydrochloride, labetalol hydrochloride, manidipine hydrochloride, candesartan, cilexetil, methyldopa, losartan, valsartan, eposartan, irbesartan, tasosartan, telmisartan and the like. As examples of the vasoconstrictors, there are phenylephrine hydrochloride and the like. As the coronary (vaso)dilators, there are carbocromen hydrochloride, molsidomin, verapamil hydrochloride and the like. As the peripheral vasodilators, there are cinnarizine and the like. As examples of the antihyperlipidemics, there are cerivastatin, simvastatin, pravastatin and the like. As the cholagogues, there are dehydrocholic acid, trepibutone and the like. The antibiotics include cefalexin, cefaclor, amoxicillin, pivmecillin hydrochloride, cefotiam hexetil hydrochloride, cefadroxil, cefixime, cefditoren pivoxil, cefteram pivoxil, cefpodoxime proxetil, cefotiam hydrochloride, cefozopran hydrochloride, cefmenoxime hydrochloride, cefsulodin sodium and other such cephems, ampicillin, ciclacillin, sulbenicillin sodium, nalidixic acid, enoxacin and other synthetic antibacterial drugs, carumonam sodium and other such monobactam, penem and carbapenem type antibiotics. Examples of the chemotherapeutic drugs are sulfamethizole, sulfamethizole hydrochloride, thiazosulfone and the like. Examples of the drugs for diabetes are tolbutamide, voglibose, pioglitazone hydrochloride, glibenclamide, troglitazone, rosiglitazone maleate, acarbose, miglitol, emiglitol and the like. As examples of the drugs for osteoporosis, there are ipriflavone and the like. Examples of the skeletal muscle relaxants are methocarbamol and the like. Examples of the antirheumatics are methotrexate, bucillamine and the like. As examples of the hormone agents, there are liothyronine, dexamethasone sodium phosphate, prednisolone, oxendrone, leuprorelin acetate and the like. Examples of the alkaloid narcotics are opium, morphine hydrochloride, ipecac, oxycodone hydrochloride, opium alkaloid hydrochloride, cocaine hydrochloride and the like. Examples of the sulphur drugs are sulfamin, sulfisomidine, sulfamethizole and the like. As examples of the remedies for gout, there are allopurinol, colchicine and the like. As examples of the anticoagulants, there are dicoumarol and the like. As examples of the anticancer drugs, there are 5-fluorouracil, uracil, mitomycin and the like. Examples of the drugs for treating Alzheimer's disease are idebenone, vinpocetine and the like. Ibuprofen, which is an antipyretic, analgesic and anti-inflammatory drug, and propranolol hydrochloride, which is a drug for arrhythmia, are examples of drug components advantageously employed in the present inventions. Two or more of the aforesaid medicinal components may be jointly incorporated into the inventive disintegrating buccal tablets. The amount of aforesaid medicinal component is normally 0.01 to 90 parts by weight, preferably 0.02 to 60 parts by weight, and more preferably 0.05 to 50 parts by weight, per 100 parts by weight of the disintegrating buccal tablets. The polyvinyl alcohol/polyethylene glycol graft copolymer used in the present inventions is a graft copolymer comprising polyvinyl alcohol units and polyethylene glycol units, and it functions as a disintegrating agent in the inventive disintegrating buccal tablets.

The aforesaid polyvinyl alcohol/polyethylene glycol graft copolymer preferably has a weight ratio of polyvinyl alcohol units to polyethylene glycol units in the range from 60:40 to 90:10, and a weight average molecular weight in the range 30,000 to 60,000. As an example of such a preferred polyvinyl alcohol/polyethylene glycol graft copolymer, there is Kollicoatm IR (75 wt % polyvinyl alcohol and 25 wt % polyethylene glycol units; weight average molecular weight about 45,000) which is marketed by BASF, and this can be used in the present inventions but other products may also be employed. From 1 to 20 parts by weight, preferably 3 to 20 parts by weight, and more preferably 5 to 20 parts by weight, of the polyvinyl alcohol/polyethylene glycol graft copolymer is used per 100 parts by weight of the disintegrating buccal tablets. Besides the physiologically-active material and the polyvinyl alcohol/polyethylene glycol graft copolymer, the inventive disintegrating buccal tablets may also include the binders, fillers/excipients, disintegrating agents, lubricants, colouring agents, acid flavourings, foaming agents, artificial sweeteners, fragrances and the like generally used in the production of disintegrating buccal tablets. The amounts added are the quantities normally used in the production of disintegrating buccal tablets. Examples of the binding agents are hydroxypropyl cellulose, hydroxypropyl methyl cellulose, crystalline cellulose, pregelatinized starch, polyvinyl pyrrolidone, gum Arabic powder, gelatine, pullulan, hydroxypropyl cellulose having a low degree of substitution, and the like.

Examples of the fillers/excipients are sugars such as lactose, sucrose and sugar alcohols (for example D-mannitol); starch, corn starch, crystalline cellulose, light silica, titanium oxide and the like. Normally, from 50 to 80 parts by weight of a sugar alcohol such as D-mannitol is incorporated per 100 parts by weight of the disintegrating buccal tablets.

As disintegrating agents, there can be employed the disintegrating agents generally used in the field of pharmaceutical preparations, examples of which are (1) crospovidone, (2) croscarmellose sodium, carmellose calcium or other such disintegrating agent known as a super disintegrant, (3) sodium carboxymethylstarch, (4) hydroxypropyl cellulose with a low degree of substitution, and (5) corn starch. The aforesaid crospovidone includes the material known as polyvinyl polypyrrolidone (PVPP) or 1-vinyl-2-pyrrolidinone homopolymer, and may be any crosslinked polymer of chemical name 1-ethenyl-2-pyrollidinone homopolymer, a specific example being Kollidon CL (produced by BASF). These disintegrating agents may be used on their own or two or more may be jointly employed.

The polyvinyl alcohol/polyethylene glycol graft copolymer used in the present inventions functions as a disintegrating agent, so it is not necessary to employ an aforesaid disintegrating agent. However, amongst the aforesaid disintegrating agents, there is preferably used from 1 to 5 parts by weight of Kollidon CL (produced by BASF) per 100 parts by weight of the disintegrating buccal tablets.

Examples of the lubricating agents are magnesium stearate, sucrose fatty acid esters, polyethylene glycol, talc, stearic acid and the like. Examples of the colouring agents are food dyes such as Food Yellow No. 5, Food Red No. 2 and Food Blue No. 2; food lake pigments, red ochre and the like. The acid flavourings include citric acid (anhydrous citric acid), tartaric acid, malic acid and the like. An example of the foaming agents is sodium bicarbonate. Examples of artificial sweeteners are saccharin sodium, dipotassium glycyrrhetate, aspartame, stevia, thaumatin and the like. The fragrances may be synthetic or natural, and examples include lemon, lime, orange, menthol, strawberry and the like

The inventive disintegrating buccal tablets can be produced by subjecting to granulation a composition containing, for example, the physiologically active material, the polyvinyl alcohol/polyethylene glycol graft copolymer and, optionally, aforesaid binder, filler/excipient, disintegrating agent and the like, after which mixing is optionally performed with a lubricant, etc, and then tableting is carried out.

The granular material produced is not particularly restricted in terms of granular form and it may, or may not, contain a core. Furthermore, in the case where the granular material has a core, said core may or may not contain a physiologically active material. Where the granular material does not have a core, it can be produced by well-known granulation methods. Examples of the granulation methods are tumbling granulation methods (for example the centrifugal tumbling granulation method), fluidized granulation methods (for example tumbling fluidized bed granulation or fluidized granulation) and stirred granulation methods. Of these, the fluidized granulation methods are preferred. The tumbling fluidized bed granulation method is particularly preferred. As a specific example of the tumbling granulation methods, there is the method using the “CF Equipment” produced by the Freund Corporation. As examples of the tumbling fluidized bed granulation method, there are the methods using for example “Spir-aFlow”, or the “Multiplex” produced by the Powrex Corporation, or the “New Marume” produced by the Fuji Paudal Co. With regard to the mixed liquid spray method, suitable selection can be made according to the type of granulation equipment and, for example, there can be used a top spray system, a bottom spray system or a tangential spray system. Of these, the tangential spray system is preferred.

On the other hand, where the granular material does have a core, production can be carried out by coating the core with the physiologically active material, etc, by a known method. For example, based on the production method (coating method) described in JP-A-5-092918, production can be carried out by coating the physiologically active material and optional binder, lubricant and filler/excipient, etc, onto a core comprising crystalline cellulose and lactose.

The particle diameter of the aforesaid granular material is not particularly restricted but fine or coarse granules are preferred, and in order not to have a rough or unpleasant feel in the mouth the average particle diameter is no more than about 400 μm. The preferred average particle diameter is 200-400 μm, and more preferably 300-400 μm.

The inventive disintegrating buccal tablets are produced by a method of the kind normally used in the pharmaceutical preparations field. For example, the aforesaid granular material is mixed with optional additives such as a lubricant and/or water, and tableting carried out, and then drying is performed where desired. The mixing is conducted by a generally-used mixing method such as mixing per se, kneading, granulating or the like. Said mixing may be carried out for example using equipment such as a Vertical Granulator VG10 (produced by the Powrex Corporation), a Universal Kneader (produced by Hata Tekkosho), a Fluidized Bed Granulator LAB-1 or FD-3S (produced by the Powrex Corporation), a V-type mixer a tumbler mixer or the like. In the case, for example, where the mixing is carried out with a V-type mixer, the magnesium stearate and the granular material containing physiologically active material, D-mannitol and polyvinyl alcohol/polyethylene glycol graft copolymer are respectively introduced into a V-type mixer of desired capacity and these mixed together for 5 minutes, after which the mixture is removed and subjected to tableting.

The tableting is carried out using a single-shot tableting machine (made by Kikusui Seisakusho Ltd), a rotary tableting machine (made by Kikusui Seisakusho Ltd) or the like and, normally, the tableting is carried out at a pressure of 0.5 to 2.0 Ton/cm² and preferably 1.5 to 2.0 Ton/cm². Drying may be carried out using any of the methods generally employed for the drying of pharmaceutical preparations, such as vacuum drying, fluidized bed drying or the like.

The inventive disintegrating buccal tablets can be taken in the same way as normal disintegrating buccal tablets by chewing, etc, without water, and then swallowing. The administered dose of such disintegrating buccal tablets will differ depending on the particular physiologically active material (the medicinal component), the administration objectives and types of symptoms, etc, and should be selected from within a range such that the amount of physiologically active material administered is an effective dose.

Below, the present inventions are explained in specific terms by means of examples but the inventions are not to be restricted to these examples.

### EXAMPLE 1

### Evaluation of the Disintegration of a 2% Preparation of the Disintegrating Agent Kollidon CL

The following test was carried out to show that polyvinyl alcohol/polyethylene glycol graft copolymer has the same kind of disintegration performance as hydroxypropyl cellulose with a low degree of substitution, which is itself known to be a base ingredient with outstanding disintegration performance.

As the polyvinyl alcohol/polyethylene glycol graft copolymer, there was used Kollicoat™ IR produced by BASF. As the hydroxypropyl cellulose with a low degree of substitution, there was used LH22 (hereinafter referred to as L-HPC) produced by the ShinEtsu Chemical Co.

12 g of vitamin C, 356 g of D-mannitol, 8 g of Kollidon CL produced by BASF and 20 g of either Kollicoatm IR produced by BASF or L-HPC were introduced into the powder fluidizing region of a Multiplex (MP-01) fluidized bed granulator produced by the Powrex Corporation and, while forming a fine spray of water, a total of 300 ml was sprinkled onto the aforesaid fluidized powder at a rate of 10 ml per minute over about 30 minutes. The production conditions were set at an air supply temperature of about 90° C. and a drying air volume of 40-50 m³/hour so as to maintain the exhaust air temperature at about 30° C. A granular product developed from the powder.

After halting the spraying of the water, drying was promoted at the same air supply temperature and when the exhaust air temperature reached 40° C. the granulation process was terminated. The coarse granular product was removed from the powder fluidizing region and passed through a 20 mesh sieve. In this way, a Kollicoat™ IR granular product and a L-HPC granular product were obtained respectively.

After mixing 4 g of magnesium stearate lubricant with 396 g of the Kollicoat™ IR granular product or the L-HPC granular product, 11.3 mm, 400 mg flat tablets were produced using a tableting machine (VIRGO506SS2AZ) produced by Kikusui Seisakusho Ltd, at a tableting pressure of 0.5, 1.0, 1.5 or 2.0 Ton/cm².

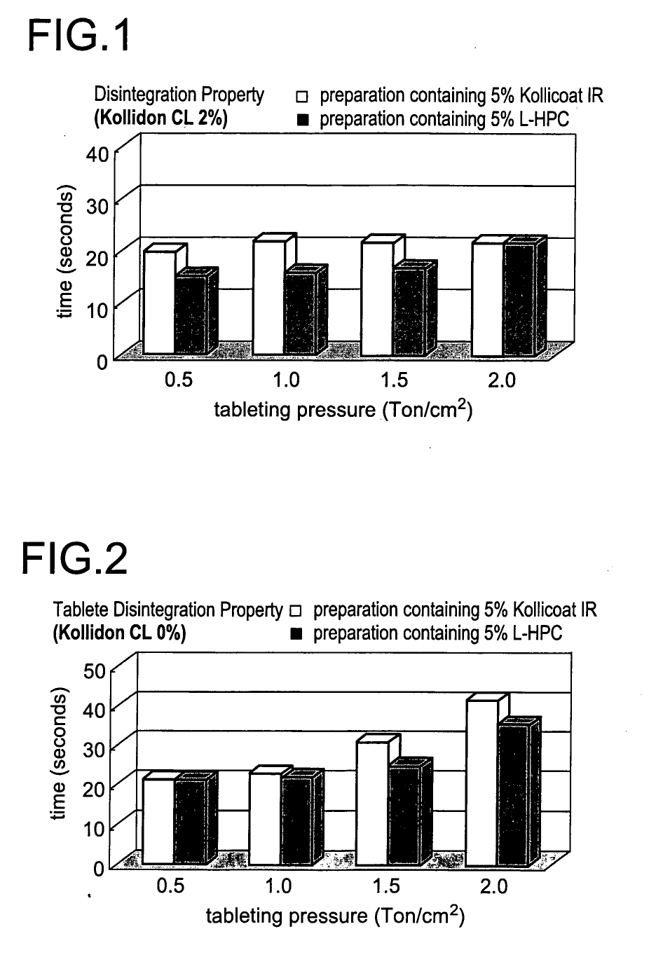

Evaluation of the disintegration properties of these flat tablets was carried out by the Japanese Pharmacopoeia disintegration test method (test liquid:water). The results are shown in FIG. 1.

Comparing the L-HPC preparation with the Kollicoat™ IR preparation, the disintegration time of the former tended to be shorter but the time was within the error range, so the disintegration performance was essentially the same.

### EXAMPLE 2

### Evaluation of the Disintegration of a Preparation Produced without Addition of the Disintegrant Kollidon CL

An evaluation was carried out of the disintegration performance of the polyvinyl alcohol/polyethylene glycol graft polymer in the case of the same formulations but with the Kollidon CL, which is said to be outstanding in its disintegration performance, excluded.

12 g of vitamin C, 364 g of D-mannitol and 20 g of either Kollicoat™ IR produced by BASF or L-HPC were introduced into the powder fluidizing region of a Multiplex (MP01) fluidized bed granulator produced by the Powrex Corporation and, while forming a fine spray of water, a total of 300 ml was sprinkled onto the aforesaid fluidized powder at a rate of 10 ml per minute over about 30 minutes. The production conditions were set at an air supply temperature of about 90° C. and a drying air volume of 40-50 m³/hour so as to maintain the exhaust air temperature at about 30° C. A granular product developed from the powder.

After halting the spraying of the water, drying was promoted at the same air supply temperature and when the exhaust air temperature reached 40° C. the granulation process was terminated. The coarse granular product was removed from the powder fluidizing region and passed through a 20 mesh sieve. In this way, a Kollicoat™ IR granular product and a L-HPC granular product were obtained respectively.

After mixing 4 g of magnesium stearate lubricant with 396 g of the Kollicoatm IR granular product or the L-HPC granular product, 11.3 mm, 400 mg flat tablets were produced using a tableting machine (VIRGO506SS2AZ) produced by Kikusui Seisakusho Ltd, at a tableting pressure of 0.5, 1.0, 1.5 or 2.0 Ton/cm².

Evaluation of the disintegration properties of these flat tablets was carried out by the Japanese Pharmacopoeia disintegration test method (test liquid:water). The results are shown in FIG. 2.

Even without the addition of Kollidon CL disintegrant, the disintegration property of the Kollicoa™ IR granular product and the L-HPC granular product was extremely good.

Kollicoat™ IR is a synthetic base ingredient with disintegrating performance matching that of L-HPC, which is a cellulose material, and its usefulness as a disintegrant for disintegrating buccal tablets is confirmed.

[FIG. 1] This shows the results of an evaluation of the disintegration property of the preparation containing 2% Kollidon CL disintegrant in Example 1.

[FIG. 2] This shows the results of an evaluation of the disintegration property of the preparation containing no Kollidon CL disintegrant in Example 2.

1-7. (canceled)

8. A disintegrating buccal tablet comprising a physiologically active material and a polyvinyl alcohol/polyethylene glycol graft copolymer.

9. The disintegrating buccal tablet according to claim 8, further comprising a sugar compound.

10. The disintegrating buccal tablet according to claim 9, wherein the sugar compound comprises a sugar alcohol.

11. The disintegrating buccal tablet according to claim 8, wherein the polyvinyl alcohol/polyethylene glycol graft copolymer comprises polyvinyl alcohol units and polyethylene glycol units in a weight ratio of 60:40 to 90:10.

12. The disintegrating buccal tablet according to claim 8, wherein the polyvinyl alcohol/polyethylene glycol graft copolymer has a weight average molecular weight of 30,000 to 60,000.

13. The disintegrating buccal tablet according to claim 11, wherein the polyvinyl alcohol/polyethylene glycol graft copolymer has a weight average molecular weight of 30,000 to 60,000.

14. The disintegrating buccal tablet according to claim 8, wherein the polyvinyl alcohol/polyethylene glycol graft copolymer is present in an amount of 1 to 20 parts by weight per 100 parts by weight of the tablet.

15. The disintegrating buccal tablet according to claim 11, wherein the polyvinyl alcohol/polyethylene glycol graft copolymer is present in an amount of 1 to 20 parts by weight per 100 parts by weight of the tablet.

16. The disintegrating buccal tablet according to claim 13, wherein the polyvinyl alcohol/polyethylene glycol graft copolymer is present in an amount of 1 to 20 parts by weight per 100 parts by weight of the tablet.

17. A disintegrating buccal tablet comprising a physiologically active material, a sugar compound and a polyvinyl alcohol/polyethylene glycol graft copolymer; wherein the polyvinyl alcohol/polyethylene glycol graft copolymer comprises polyvinyl alcohol units and polyethylene glycol units in a weight ratio of 60:40 to 90:10; wherein the polyvinyl alcohol/polyethylene glycol graft copolymer has a weight average molecular weight of 30,000 to 60,000; and wherein the polyvinyl alcohol/polyethylene glycol graft copolymer is present in an amount of 1 to 20 parts by weight per 100 parts by weight of the tablet.

18. A method comprising:
(a) granulating a composition comprising a physiologically active material and a polyvinyl alcohol/polyethylene glycol graft copolymer; and
(b) tableting the granulated composition.

19. The method according to claim 18, wherein the composition further comprises a sugar compound.

20. The method according to claim 19, wherein the sugar compound comprises a sugar alcohol.

21. The method according to claim 18, wherein the polyvinyl alcohol/polyethylene glycol graft copolymer comprises polyvinyl alcohol units and polyethylene glycol units in a weight ratio of 60:40 to 90:10.

22. The method according to claim 18, wherein the polyvinyl alcohol/polyethylene glycol graft copolymer has a weight average molecular weight of 30,000 to 60,000.

23. The method according to claim 21, wherein the polyvinyl alcohol/polyethylene glycol graft copolymer has a weight average molecular weight of 30,000 to 60,000.

24. The method according to claim 18, wherein the polyvinyl alcohol/polyethylene glycol graft copolymer is present in an amount of 1 to 20 parts by weight per 100 parts by weight of the tablet formed.

25. The method according to claim 21, wherein the polyvinyl alcohol/polyethylene glycol graft copolymer is present in an amount of 1 to 20 parts by weight per 100 parts by weight of the tablet formed.

26. The method according to claim 23, wherein the polyvinyl alcohol/polyethylene glycol graft copolymer is present in an amount of 1 to 20 parts by weight per 100 parts by weight of the tablet formed.

27. A disintegrating buccal tablet prepared by a method according to claim 18.
